(19) United States
(12) Patent Application Publication
(10) Pub. No.: US 2025/0078543 A1
(43) Pub. Date: Mar. 6, 2025

(54) METHOD FOR DETERMINING THE STATUS OF GREASE CONTAINING PARTICLES BY IMAGE RECOGNITION

(71) Applicant: Aktiebolaget SKF, Göteborg (SE)

(72) Inventors: Weichen GU, Shanghai (CN); Jinshi CUI, Shanghai (CN)

(21) Appl. No.: 18/808,999

(22) Filed: Aug. 19, 2024

(30) Foreign Application Priority Data
Aug. 30, 2023 (CN) 202311109489.1

(51) Int. Cl. G06V 20/69; G01N 1/28; G01N 15/0227; G01N 21/88; G06V 10/82; G08B 21/18

(52) U.S. Cl. CPC G06V 20/693; G01N 1/2813; G01N 15/0227; G01N 21/8851; G06V 10/82; G06V 20/698; G01N 2021/8854; G01N 2021/8887; G08B 21/182

(57) ABSTRACT

A method for determining the status of grease containing particles by image recognition. The method may include sampling the grease and spreading the grease on a filter membrane. The method may also include image recording of the sampled grease by a microscope, which may include: setting a magnification of the microscope, setting a distance of a lens of the microscope to the filter membrane, and/or recording an image of the sampled grease by the microscope. The method may also include performing image recognition in the image by artificial intelligence and determining a particle content in the grease. The method may also include determining the status of the grease based on the particle content.

### CROSS-REFERENCE TO RELATED APPLICATION

This application claims priority to Chinese Application No. 202311109489.1, filed Aug. 30, 2023, the entirety of which is hereby incorporated by reference.

### FIELD

The present disclosure relates to a method for determining the status of grease containing particles by image recognition.

### BACKGROUND

In the operation of machinery, it is desirable to lubricate the running machinery with grease to reduce friction between parts in the machinery, thereby improving the performance of the machinery and enabling protection of the machinery. Therefore, for a better lubricating effect, the lubricating grease needs to maintain a good grease status. Conversely, poor grease status can lead to early operational anomalies and later failure or damage to the machinery.

Therefore, it is important to understand the grease status of lubricating grease. Conventional detection methods require specialized detection equipment or require long detection times. There is also a need to develop a new detection method in order to more simply and quickly determine the grease status of lubricating grease.

### SUMMARY

The present disclosure provides a method for determining the status of grease containing particles by image recognition. The method according to the present disclosure can improve the image recording quality of the grease image when the grease status of the lubricating grease is detected by image recognition at the site of the device, thereby improving the speed and accuracy of the image recognition. The status of the grease or the degree of contamination of the grease can thereby be determined quickly and accurately, so that corresponding maintenance measures can be taken as early as possible.

The present disclosure provides a method for determining a status of grease containing particles by image recognition, the method including: sampling the grease and spreading the grease on a filter membrane; Image recording of the sampled grease by a microscope, including: setting a magnification of the microscope, setting a distance of a lens of the microscope to the filter membrane, and recording an image of the sampled grease by the microscope; Performing image recognition in the image by artificial intelligence and determining a particle content in the grease; And determining the status of the grease based on the particle content.

In an embodiment according to the present disclosure, determining the status of the grease according to the particle content includes: Setting a plurality of particle content intervals in succession, wherein the particle content intervals classify the particle content, a larger numerical particle content interval corresponding to a larger grease contamination range; And determining a grease contamination level according to the particle content interval in which the particle content is located.

In an embodiment according to the present disclosure, in a case where the sampled grease is subjected to a plurality of image recordings by a microscope, if two of the respective particle contents determined based on the plurality of image recordings are located in non-adjacent particle content intervals, the determination of the particle contents is assumed to be invalid, and if the respective particle contents determined based on the plurality of image recordings are located in adjacent particle content intervals, the grease contamination level is determined according to the particle content interval in which the majority of the particle contents are located.

In an embodiment according to the present disclosure, a grease contamination indication is provided according to the grease contamination level, wherein the grease contamination indication includes: grease normal, contamination warning, and severe contamination.

In an embodiment according to the present disclosure, image recording of the sampled grease by a microscope further includes: making particles in the grease appear black with polarized light.

In an embodiment according to the present disclosure, image recording of the sampled grease by the microscope further includes: fixing a position of the microscope relative to the sampled grease with a fixing mount.

In an embodiment according to the present disclosure, the microscope magnification is between 100 and 500 times.

In an embodiment according to the present disclosure, the filter membrane remains flat and is placed horizontally.

In an embodiment according to the present disclosure, the filter membrane is a 5 μm to 40 μm filter membrane.

In an embodiment according to the present disclosure, the microscope is a portable microscope.

In an embodiment according to the present disclosure, image recording of the sampled grease by a microscope, further includes: judging whether image quality of the image can be used for image recognition, wherein only in a case that the image quality complies with a requirement, image recognition is performed in the image by artificial intelligence and particle content in the grease is determined; And determining the status of the grease based on the particle content.

In an embodiment according to the present disclosure, performing image recognition in the image by artificial intelligence and determining a particle content in the grease includes: determining a particle-related characteristic value in the image by artificial intelligence, and determining the particle content according to the particle-related characteristic value, wherein the particle-related characteristic value includes: a statistical characteristic value of values of pixels in the image, a number, a density and a total area of the particles.

The microscope as well as the image recording manner can be reasonably adjusted and set up by the method according to the present disclosure to record images or photographs of the sampled grease with high quality. With the teachings of the method according to the present disclosure, the grease status of the lubricating grease can be quickly and accurately determined at the plant site. Therefore, the maintenance personnel of the machine can easily and quickly perform the test in the field, and can immediately judge whether or not the grease needs to be replaced, so that it is not necessary to bring the sampled grease back to the laboratory for the test, and it is not necessary to perform the test using a specific test apparatus in the laboratory.

### BRIEF DESCRIPTION OF THE DRAWINGS

In order to more clearly illustrate the technical solutions of the embodiments of the present disclosure, the following will simply introduce the accompanying drawings that are needed to be used in the description of the embodiments. The drawings in the following description are merely exemplary embodiments of the present disclosure.

FIG. 1 illustrates a flow chart of a method for determining a status of grease containing particles by image recognition according to an embodiment of the present disclosure,

FIG. 2 illustrates a schematic view of a grease image that is available for detection according to an embodiment of the present disclosure, and

FIG. 3 illustrates a schematic view of a grease image that is not available for detection according to an embodiment of the present disclosure.

### DETAILED DESCRIPTION

In order to make the objects, technical solutions, and advantages of the present disclosure more apparent, example embodiments according to the present disclosure will be described in detail below with reference to the accompanying drawings. Apparently, the described embodiments are only a part of the embodiments of the present disclosure, rather than all of the embodiments of the present disclosure, it is understood that the present disclosure is not limited by the example embodiments described herein.

In the present specification and drawings, substantially the same or similar steps and elements are denoted with the same or similar reference numerals, and repeated description of these steps and elements will be omitted. Meanwhile, in the description of the present disclosure, the terms “first,” “second,” and the like are used only to distinguish descriptions and are not to be understood as indicating or implying relative importance or ordering.

In the present specification and drawings, elements are described in singular or plural form according to an embodiment. However, the singular and plural forms are appropriately selected for the proposed cases merely for convenience of explanation and are not intended to limit the present disclosure thereto. Thus, a singular form may include a plural form, and a plural form may include a singular form as well, unless the context clearly indicates otherwise.

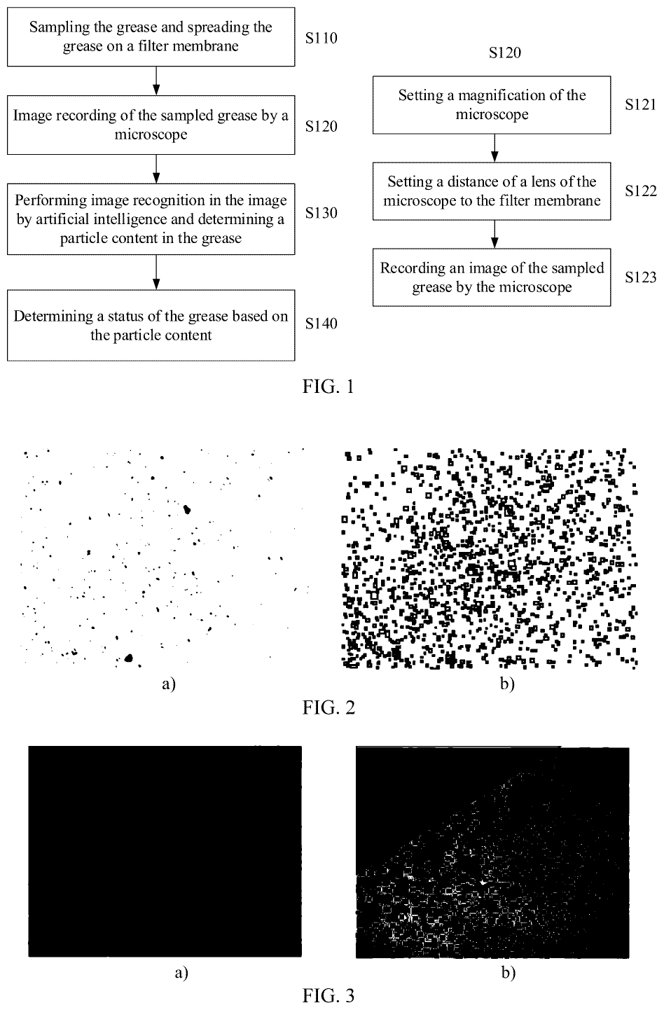

FIG. 1 illustrates a flow diagram of a method 100 for determining a status of grease containing particles by image recognition, in accordance with an embodiment of the present disclosure. The method 100 includes: sampling the grease and spreading the grease on a filter membrane (step S110); Performing image recording of the sampled grease by a microscope (step S120), performing image recognition in the image by artificial intelligence and determining the particle content in the grease (step S130); And the status of the grease is determined based on the particle content (step S140).

In this embodiment, step S120 may include, e.g., setting a magnification of the microscope (step S121), setting a distance of a lens of the microscope to the filter membrane (step S122), and recording an image of the sampled grease by the microscope (step S123).

In an embodiment according to the present disclosure, the grease is in particular a lubricating grease in a device, such as a device with a bearing, in particular a wind generator with a giant bearing. Lubricating grease is required to be added to the bearing for proper operation of the equipment, reduced wear and extended life. Despite the addition of grease, the operation of the bearing is subject to wear, whereby metal particles, in particular iron particles, are present in the grease. Therefore, the analysis of the content of iron particles in the grease is an important indicator for detecting whether maintenance or replacement of the grease is required and whether the bearing is malfunctioning.

Traditional grease testing methods require that the grease be taken to a laboratory for testing after it has been sampled. This detection method requires a long detection time due to long distance transport and experimental waiting time. This results in no timely understanding of the grease status and of the corresponding equipment, which may increase the risk of damage to the equipment and further increase maintenance costs.

Methods according to embodiments of the present disclosure may directly determine grease status through image recognition at the device site without the need to bring grease to a laboratory. In method step S110, e.g., grease in the device may be sampled and spread on the filter membrane. In an embodiment according to the present disclosure, the filter membrane may be, e.g., a 5 μm to 40 μm filter membrane for particles in the grease, e.g. iron particles, to penetrate the filter membrane and facilitate subsequent viewing and image recording. The grease is then spread on the filter membrane, preferably uniformly, and may be within 3 mm, preferably not more than 2 mm in thickness. In addition, it is preferable to keep the filter membrane flat and to be placed horizontally, otherwise the subsequently observed and recorded images appear blurred, thereby affecting the recognition effect of the iron particles.

In method step S120, the sampled grease is image recorded by a microscope. The microscope may be, e.g., a portable microscope to facilitate viewing and recording of the grease image at the site of the apparatus. In an embodiment according to the present disclosure, e.g., the magnification of the magnifying glass may first be adjusted to clearly observe the particles in the grease. Too high a magnification leads to an enlargement of the profile of the filter membrane, which affects the subsequent identification of iron particles, and too low a magnification leads to an inability to distinguish between background and iron particles. The magnification is preferably between 100 and 500 times. At the same time, it is also possible, e.g., to set the distance of the lens of the microscope to the filter membrane in order to clearly observe the particles in the grease. In an embodiment according to the present disclosure, a fixing mount may also be utilized to fix the position of the microscope relative to the sampled grease, thereby ensuring that no wobble occurs at the time of image recording by the microscope. In an embodiment according to the present disclosure, polarized light is also utilized to cause the particles in the grease to appear black to facilitate subsequent observation and identification of the iron particles.

By some or all of the method steps described above, a clear image can be obtained when an image of the sampled grease is recorded by a microscope, and subsequently the image can be subjected to image recognition by artificial intelligence and the particle content in the grease is determined. FIG. 2 illustrates a schematic view of a grease image that is available for detection according to an embodiment of the present disclosure. The plot a) in FIG. 2 illustrates an image recording of a grease containing particles recorded by a microscope. The iron particles can be clearly seen in this image and are uniformly distributed in the image. This image is available for subsequent artificial intelligence identification to determine the particle content in the grease. The plot b) in FIG. 2 illustrates the iron particles identified by artificial intelligence, which are very clear and homogeneous.

FIG. 3 illustrates a schematic view of a grease image that is not available for detection according to an embodiment of the present disclosure. The upper left corner of the plot a) in FIG. 3 is blurred compared to the plot a) in FIG. 2. This may be due to an improper distance from the lens of the microscope to the filter membrane, an uneven filter membrane, or an uneven spread of grease. The plot b) in FIG. 3 illustrates iron particles identified by artificial intelligence. It can be seen from this image that no iron particles can be identified in the upper left corner of the image. Thus, plot a) in FIG. 3 cannot be used for subsequent image recognition. In this case, it is necessary to re-image the sampled grease, or to re-sample the grease. In an embodiment according to the present disclosure, image recording of the sampled grease by a microscope, further includes: judging whether image quality of the image can be used for the image recognition, wherein only in a case that the image quality complies with a requirement, image recognition is performed in the image by artificial intelligence and particle content in the grease is determined; And determining the status of the grease based on the particle content.

In an embodiment according to the present disclosure, in case there is a plurality of grease images, it is for example necessary to name these grease images, where the naming may include: e.g.: device model, device location, device unit number, device part location, sampling point, and sampling time. The grease image may be named, e.g., according to “device model-device location-device unit number-device part location-sampling point-sampling time”. In the case where the device is a wind turbine, the naming manner can be used as follows: “wind turbine model-wind farm name-wind turbine unit number-bearing position-sampling point-sampling time”.

In an embodiment according to the present disclosure, the artificial intelligence may be implemented, e.g., by artificial intelligence algorithms such as deep learning algorithms, convolutional neural networks, etc., which determine the particle content in the grease by identifying the particles themselves in the grease, or with respect to characteristics of the particles. In an embodiment according to the present disclosure, the image recognition function of artificial intelligence may be implemented both in a device, in particular a carry-on device, e.g., the image recognition function of artificial intelligence may be integrated into a portable microscope. Alternatively, the image recognition function of artificial intelligence may also be implemented on a server in the cloud. In this case, the image of the grease recorded by the microscope can be directly uploaded to the server in the cloud in the form of data, or the image of the grease magnified by the microscope can be taken with a camera and subsequently uploaded to the server in the cloud, e.g., by means of an application.

In an embodiment according to the present disclosure, performing image recognition in the image by artificial intelligence and determining a particle content in the grease includes: determining a particle-related characteristic value in the image by artificial intelligence, and determining the particle content according to the particle-related characteristic value.

In an embodiment according to the present disclosure, the particle-related characteristic value includes a statistical characteristic value of the values of the pixels in the image, the number, density and total area of the particles.

An image may be considered to be, e.g., a collection of pixels, which each have a corresponding value. E.g., for a grayscale image, the higher the value of a pixel, the whiter/lighter the color of that pixel, whereas, the smaller the value of a pixel, the darker/darker the color of that pixel. Further, e.g., in an RGB (red, green, blue) color image, the value of the pixel may be the value of the pixel in one of the color channels. Thus, if the value of the pixel is larger, the color is more intense and vice versa the color is less intense. Alternatively, e.g., in an HSV (hue, saturation, lightness) color image, the value of the pixel may be the value of the pixel in one of the channels. Thus, for a hue, the value of a pixel represents the corresponding color; For saturation, the value of the pixel represents a high degree of color purity; For luminance, the value of a pixel represents the brightness of a color. Since the image contains particles, the size of the values of the pixels in the image may be affected by the particles, e.g. the pixels constituting the particles may have larger values. Thus, e.g., a statistical analysis may be performed for the values of the pixels and a statistical characteristic value of the values of the pixels determined.

Furthermore, after obtaining the image containing the particles, the particles may be identified from the image, e.g. the number of particles may be directly used as the characteristic value for the particles, or the density of the particles in the image may be calculated from the number of particles and the size of the image and used as the characteristic value for the particles, or the areas of all the particles may be summed to obtain the total area of the particles and used as the characteristic value for the particles.

The particle-related characteristic value has a certain connection with the particle content in the grease so that the particle content can be determined based on the particle-related characteristic value. The relationship of the particle-related characteristic value to the particle content in the grease may be established, e.g., through artificial intelligence, such as an artificial neural network.

In an embodiment according to the present disclosure, determining the status of the grease according to the particle content (step S140) may include, e.g.: continuously setting a plurality of particle content intervals, wherein the particle content intervals classify the particle content, a larger numerical particle content interval corresponding to a larger grease contamination range; And determining the grease contamination level according to the particle content interval in which the particle content is located.

In an embodiment according to the present disclosure, 2 or more particle content intervals may be set, e.g., 5 particle content intervals may be set, e.g. [0-first threshold ppm], [first threshold ppm-second threshold ppm], [second threshold-third threshold ppm], [third threshold ppm-fourth threshold ppm], [>fourth threshold ppm], wherein ppm (parts per million) is a unit of iron particle content in commonly used lubricating grease. These particle content intervals represent different grades of particle content in the grease, where the particle content intervals correspond to grades I, II, III, IV and V, respectively. A higher grade represents a higher level of particle content in the grease, i.e. the grease is more contaminated.

In an embodiment according to the present disclosure, a grease contamination indication may be provided according to the grease contamination level, wherein the grease contamination indication includes: grease normal, contamination warning, and severe contamination. As shown in the table below, for the particle content interval corresponding to grade I, the grease can be considered to be in a normal state; For the particle content interval corresponding to grade II and III, the lubricating grease can be considered to be lightly contaminated and is therefore in a contamination warning state; For the particle content interval corresponding to grade IV and V, the lubricating grease can be considered to be in a heavily contaminated state.

| Particle Content Interval (ppm) | Grade | Grease status |
|---|---|---|
| 0-first threshold ppm | I | grease normal |
| first Threshold-second | II | Contamination warning |
| Threshold ppm | | |
| second Threshold-third | III | Contamination warning |
| Threshold ppm | | |
| third Threshold-fourth | IV | severe contamination |
| Threshold ppm | | |
| >fourth threshold ppm | V | severe contamination |

In the embodiments according to the present disclosure, in order to determine the particle content in the grease as accurately as possible, image recording as well as image recognition may be performed multiple times, preferably 3 times, 5 times, 7 times or more. If the same result is obtained through multiple image recordings and corresponding image recognition, e.g., the determined particle content is located in the same particle content interval, the particle content grade or degree of contamination or grease status can be determined based on the corresponding particle content interval. If each particle content determined based on the plurality of image recordings located in a different particle content interval, it needs to be taken into account case by case. In the first case, if two of the determined particle contents lie in non-adjacent particle content intervals, the determination of the particle contents is assumed to be invalid. E.g., one of the particle contents is located in [0-first threshold ppm] and the other particle content is located in [second threshold-third threshold ppm], in which case the determined particle contents are considered to deviate too much and thus the determination of the particle content is ineffective. In the second case, if the respective particle contents determined based on the plurality of image recordings are located in adjacent particle content intervals, the grease contamination level is determined based on the particle content interval in which the majority of the particle contents are located.

In an embodiment according to the present disclosure, in a case where the particle content is divided into 5 particle content intervals and thus into 5 grades, the grease contamination level may be determined, e.g., according to a rule as in the following table.

According to the following table, in the case where image recording is performed on grease a plurality of times, and image recognition is performed on these grease images by artificial intelligence and particle contents in the grease are respectively determined, if all the determined particle contents are grade I, it can be directly determined that the grease state is normal, and no processing of the grease is required. If the determined particle content includes grade I and II, the grease status is determined based on the occurrence of a majority or a high probability. E.g., when the majority is grade I, the grease is determined to be normal and no further processing is required; And when the majority is grade II, the grease status is determined to be a contamination warning and the level of particles in the grease can be further determined in other ways, e.g., the grease can be brought back to the laboratory for more accurate detection. If the determined particle content includes grade II and/or grade III and grade IV, the grease status is determined based on a majority occurrence or a high probability of occurrence. E.g., when the majority is grade II and/or grade III, the grease status is determined to be a contamination warning and other means may be taken to further determine the particle content in the grease; and when the majority is grade IV, the grease status can be determined to be severely contaminated, requiring cleaning or replacement of the grease. When the determined particle content includes grade II and/or grade III and grade V or includes grade IV and/or grade V, it can be directly determined that the grease status is severely contaminated, requiring cleaning or replacing of grease. In addition, when the determined particle content includes both grade I and grade IV and/or grade V, the result of the determined particle content is considered to deviate too much, i.e., the result is not valid. In this case, it is necessary to re-determine the particle content or re-collect the grease.

| Determined particle content | | |
|---|---|---|
| (From Grease Images) | Rules | Mode of Treatment |
| Including only grade I | Determined to be grease normal | No further treatment |
| Including grade I | Grease is determined to be normal | No further treatment |
| and grade II | when the majority is grade I | |
| | (high probability is grade I) | |
| | A contamination warning is | Further determination |
| | determined when the majority | of particle content in |
| | is grade II (large probability | grease by other means |
| | is grade II) | |
| Including grade II | Determined as a contamination | Further determination |
| and/or grade III | warning when the majority is | of particle content in |
| and grade IV | grade II and/or grade III | grease by other means |
| | Determined to be severely | Cleaning or replacing |
| | contaminated when the majority | of grease required |
| | is grade IV | |
| Including grade II | Determined to be severely | Cleaning or replacing |
| and/or grade III | contaminated | of grease required |
| and grade III | | |
| Including grade IV | Determined to be severely | Cleaning or replacing |
| and/or grade V | contaminated | of grease required |
| Including grade I and | Determined to be Invalid | Re-determination of |
| grade IV and/or grade V | | particle content or re- |
| | | collection of grease |

The microscope as well as the image recording manner can be reasonably adjusted and set up by the method according to the present disclosure to record images or photographs of the sampled grease with high quality. With the teachings of the method according to the present disclosure, the grease status of the lubricating grease can be quickly and accurately determined at the plant site. Therefore, the maintenance personnel of the machine can easily and quickly perform the test in the field, and can immediately judge whether or not the grease needs to be replaced, so that it is not necessary to bring the sampled grease back to the laboratory for the test, and it is not necessary to perform the test using a specific test apparatus in the laboratory.

The method according to the present disclosure may enable the determination of the particle content, e.g., iron particles, in a fluid to be detected, e.g., a grease, by means of only an image of the fluid. Therefore, the maintenance personnel of the machine can easily and quickly perform the test in the field, and can immediately judge whether or not the grease needs to be replaced, so that it is not necessary to bring the sampled grease back to the laboratory for the test, and it is not necessary to perform the test using a specific test apparatus in the laboratory.

The block diagrams of circuits, units, devices, apparatuses, devices, systems referred to in this disclosure are merely illustrative examples and are not intended to require or imply that the connections, arrangements, configurations must be made in the manner shown in the block diagrams. As will be appreciated by those skilled in the art, these circuits, units, devices, apparatuses, devices, systems may be connected, arranged, configured in any way as long as the desired purpose is achieved. The circuits, units, devices, apparatuses involved in the present disclosure may be implemented in any suitable manner, e.g., in an Application Specific Integrated Circuit, a Field Programmable Gate Array (FPGA) or the like, or in a general-purpose processing unit in combination with a known program.

It should be understood by those skilled in the art that the above-described specific embodiments are only examples and not limitations, and various modifications, combinations, partial combinations and substitutions may be made to the embodiments of the present disclosure according to design requirements and other factors as long as they are within the scope of the appended claims or the equivalent thereof, i.e., the scope of the right to be protected by the present disclosure.

What is claimed is:

1. A method for determining a status of grease containing particles by image recognition, the method comprising:
sampling the grease and spreading the grease on a filter membrane;
image recording of the sampled grease by a microscope, comprising:
setting a magnification of the microscope,
setting a distance of a lens of the microscope to the filter membrane, and
recording an image of the sampled grease by the microscope;
performing image recognition in the image by artificial intelligence and determining a particle content in the grease; and
determining a status of the grease based on the particle content.

2. The method of claim 1, wherein determining a status of the grease based on the particle content comprises:
setting a plurality of particle content intervals in succession, wherein the particle content intervals classify the particle content and a larger number of particle content intervals corresponds to a larger range of grease contamination; and
determining a grease contamination level according to the particle content interval in which the particle content is located.

3. The method according to claim 2, wherein in a case where the sampled grease is subjected to a plurality of image recordings by a microscope,
if two of the respective particle contents determined based on the plurality of image recordings are located in non-adjacent particle content intervals, the determination of the particle contents is assumed to be invalid,
if the respective particle contents determined based on the plurality of image recordings are located in adjacent particle content intervals, the grease contamination level is determined based on the particle content interval in which the majority of the particle contents are located.

4. The method according to claim 2, wherein a grease contamination indication is provided according to the grease contamination level, wherein the grease contamination indication comprises: grease normal, contamination warning and severe contamination.

5. The method according to claim 3, wherein a grease contamination indication is provided according to the grease contamination level, wherein the grease contamination indication comprises: grease normal, contamination warning and severe contamination.

6. The method of claim 1, wherein image recording of the sampled grease by a microscope further comprises: making particles in the grease appear black with polarized light.

7. The method of claim 1, wherein image recording of the sampled grease by a microscope further comprises fixing a position of the microscope relative to the sampled grease with a fixing mount.

8. The method of claim 1, wherein the microscope magnification is between 100 and 500 times.

9. The method of claim 1, wherein the filter membrane remains flat and is placed horizontally.

10. The method of claim 1, wherein image recording of the sampled grease by a microscope, further comprises:
judging whether the image quality of the image can be used for the image recognition,
wherein only in the case that the image quality complies with a requirement, image recognition is performed in the image by artificial intelligence and particle content in the grease is determined; and the status of the grease is determined based on the particle content.

11. The method of claim 1, wherein performing image recognition in the image by artificial intelligence and determining a particle content in the grease comprises:
determining a particle-related characteristic value in the image by artificial intelligence, and
determining the particle content according to the particle-related characteristic value,
wherein the particle-related characteristic value comprises a statistical characteristic value of the values of pixels in the image, the number, density and total area of the particles.
